(19) United States
(12) Patent Application Publication
(10) Pub. No.: US 2003/0022856 A1
(43) Pub. Date: Jan. 30, 2003

(54) Method for sustained release local delivery of drugs for ablation of unwanted tissue

(75) Inventors: Thomas Richardson, Ann Arbor, MI (US); David J. Mooney, Ann Arbor, MI (US)

Correspondence Address: MILLEN, WHITE, ZELANO & BRANIGAN, P.C., 2200 CLARENDON BLVD., SUITE 1400, ARLINGTON, VA, 22201, US

(73) Assignee: The Regents Of The University of Michigan, Ann Arbor, MI

(21) Appl. No.: 10/058,835

(22) Filed: Jan. 30, 2002

Related U.S. Application Data
(60) Provisional application No. 60/264,713, filed on Jan. 30, 2001.

(51) Int. Cl. A61K 48/00; A61K 38/19; A61K 38/17; A61K 31/704; A61K 31/7072; A61K 31/525; A61K 9/14

(52) U.S. Cl. 514/44; 514/12; 424/85.1; 514/251; 514/50; 514/33; 424/486

(57) ABSTRACT

Described herein are methods for ablation, i.e., elimination or reduction, of unwanted tissue, particularly tissue which is normal to be present in the body but is unwanted for either health or cosmetic reasons. In particular embodiments, there are described methods for elimination of fat tissue from the body. According to the methods described herein, a drug which acts to eliminate the undesired tissue is provided in a carrier which is biocompatible, capable of being administered by injection, and which effects a controlled release of the drug over time. The drug with carrier is administered by injection locally in the area of the unwanted tissue, resulting in elimination of the tissue in that local area.

This application claims benefit of U.S. provisional application no. 60/264,713, filed Jan. 30, 2001.

The U.S. Government has a paid-up license in this invention and the right in limited circumstances to require the patent owner to license others on reasonable terms as provided for by the terms of National Institutes of Health Grant No. DE07057.

Described herein are methods for ablation, i.e., elimination or reduction, of unwanted tissue, particularly tissue which is normal to be present in the body but is unwanted for either health or cosmetic reasons. In particular embodiments, there are described methods for elimination of fat tissue from the body. According to the methods described herein, a drug which acts to eliminate the undesired tissue is provided in a carrier which is biocompatible, capable of being administered by injection, and which effects a controlled release of the drug over time. The drug with carrier is administered by injection locally in the area of the unwanted tissue, resulting in elimination or reduction of the tissue in that local area.

### BACKGROUND OF THE INVENTION

Obesity represents a major public health issue that continues to grow and accounts for 5.7% of total direct health care costs in the United States, and increases the risk of many of the leading causes of death (e.g., cardiovascular disease, diabetes and cancer). Obesity is marked by excess adipose (i.e., fat) tissue accumulation arising from both an increased number of adipocytes and an increased size of adipocytes due to higher levels of lipid storage. Excess adipose tissue is strongly correlated with numerous health problems, including diabetes (e.g., decreased insulin sensitivity), vascular disease (e.g., hypertension) and certain forms of cancer. Recent reports have described rising obesity rates to be 33.4% of the adult population between 1988-1991. Citing the substantial rate increase in recent years, and the likely escalation, the Center for Disease Control described the alarming trend as an ‘epidemic’, using language previously reserved for infectious diseases. This points to a renewed interest by public health officials in combating obesity. Based on the numbers of those affected by obesity and the closer scrutiny of the health issues by policymakers, a large market currently exists for health-related obesity treatments and will undoubtedly grow. Exacerbating the health risks associated with obesity is that the most popular treatment for morbid obesity, liposuction, is a largely unregulated, half-billion dollar, industry that exposes patients to additional health risks, including infection and death. As the current strategy for adipose reduction, liposuction is an invasive and painful procedure requiring costly equipment and considerable recovery time and often results in inconsistent tissue shape.

Efforts to regulate adipose levels non-surgically have relied on the development of drugs primarily focused on regulating satiety and energy balance (e.g., leptin, uncoupling proteins), with the presumption that weight loss will occur in response to decreased food intake or increased energy expenditure. Other research efforts are aimed at understanding the development and differentiation pathways of the adipocytes, a process with numerous potential drug targets. While these promising approaches aim to decrease the health risks associated with obesity, they currently do not offer the ability to reduce specific stores of fat. The importance of this point is underscored by the pathologic functions of visceral adipose tissue associated with endocrine and metabolic malfunctions.

It has been known that certain drugs, including TNF-α, can be generally administered, as opposed to locally administered, to combat obesity; see, e.g., Shah (U.S. Pat. No. 6,020,004), Larrick et al. (U.S. Pat. No. 4,684,623), Girten et al. (U.S. Pat. No. 5,726,156) and Cincotta et al. (U.S. Pat. No. 5,344,832). Also, at least one method has been taught for accelerating local weight reduction by administration of a certain beta-adrenergic stimulator or alpha-2 adrenergic inhibitor, although not in controlled release form; see, Greenway, III et al. (U.S. Pat. Nos. 4,588,724 and 4,525,359).

Additionally, methods for local delivery of sustained release formulations of drugs have been known for other purposes; see, e.g., Silvestri et al. (U.S. Pat. No. 5,126,147) and Leibovich et al. (U.S. Pat. No. 4,808,402). Among such methods are those used in conjunction with cancerous tumor removal. But no method has been disclosed for localized administration of a sustained release drug formulation for the local elimination or reduction of normal, but undesired, tissue.

### SUMMARY OF THE INVENTION

We have developed a novel drug delivery application; the ablation of normal but unwanted tissue, such as fat tissue, by the sustained, localized release of a tissue ablation drug, e.g., tumor necrosis factor-α(TNF-α) for fat ablation. The description below in parts is described in terms of a particular method for ablation of fat tissue with the drug TNF-α. This should be taken as only exemplary since, as further discussed below, the method may be applied to elimination or reduction of other types of tissue using other types of drugs.

An aspect of the invention is that adipose tissue mass can be destroyed by sustained, targeted delivery of TNF-α. TNF-α has been implicated as the central mediator of adipose tissue mass based on its ability to induce apoptosis of adipocytes and to increase the lipolytic/lipogenic balance of the tissue, leading to an overall decrease in adipose mass. As adipose tissue develops and matures, TNF-α is expressed to serve as a homeostatic strategy to limit adipose tissue growth. TNF-α induces both lipolysis and apoptosis, while also inducing the expression of leptin, a protein that signals the level of satiety. In morbidly obese patients, TNF-α expression is lost, suggesting that adipose tissue can grow relatively unabated and without concurrent signals for satiety. The result is compromised endocrine function of the tissue and increased cardiovascular stress. Therefore, as TNF-α plays a role in mediating adipose mass, it is an ideal candidate for use therapeutically. However, TNF-α poses a challenge to therapeutic utility using typical drug delivery approaches (e.g., systemic delivery via infusion), due to its well-described effects on glucose metabolism when present in the systemic circulation. When found at sufficient levels systemically, TNF-α impairs insulin signaling, inhibits glucose clearance via downregulation of glucose transporters in muscle, and can be toxic over the long term. Thus, restricted TNF-α localization is likely of paramount importance to its therapeutic use for selective adipose ablation.

One objective of the invention was to develop drug delivery systems capable of targeted and controlled delivery of TNF-α to sites of adipose tissue accumulation to regulate obesity and provide a novel means for “spot-reduction” of fat tissue. It has been discovered that according to methods of the invention, this objective is achieved. Fat may be selectively destroyed by a local, sustained delivery of TNF-α. The use of a local, sustained TNF-α delivery can overcome the major drawbacks encountered with previous methods, particularly liposuction, with a minimally invasive injection. Further, local administration can avoid the disadvantage of possible impaired glucose intolerance (i.e., diabetes) which can be expected in a general administration, such as disclosed in the Shah patent (U.S. Pat. No. 6,020,004). A considerable advantage is achieved from the therapeutic modality described herein aimed at the safe reduction of adipose tissue mass using minimally invasive procedures.

The embodiment of the invention directed to methods for fat ablation with TNF-α effect a localized reduction of fat mass. Using a controlled, sustained release of TNF-α from a polymer, fat tissue can be destroyed in a localized manner. The loss of fat pad mass according to the invention is not due to an overall weight loss, as is shown in the following examples by comparison to a contralateral fat pad for a control in the test animals. In those examples, the TNF-α treatment resulted in an average fat pad weight of 85% that of the contralateral control. The measured loss in these examples thus being 15% due to a single injection into the fat and assayed at a single time point. Multiple injections would be predicted to mediate fat ablation within numerous regions within the tissue, and maximal fat loss could occur at longer or shorter time points. These results are considered to be very encouraging since this is the first description of a sustained, localized delivery approach to ablate fat tissue and thus, no standard treatment protocols currently exist.

Furthermore, the method may also be applied for selectively eliminating or reducing fat tissue by the sustained delivery of fat reducing factors other than TNF-α, including other drugs, DNA, anti-sense RNA, and other proteins, that are, or could be, involved in adipose homeostasis. Examples of such other factors include, but are not limited to other proteins involved in fat metabolism (e.g., uncoupling proteins, leptin, orexin, etc.); antisense RNA molecules designed to knock out the specific activity of any individual protein involved in fat cell maintenance (e.g., transcription factors, enzymes, cell cycle regulators, etc.); DNA, either in the form of plasmids or viruses, designed to induce the expression of apoptosis (cell death)-inducing factors, or other molecules that could disrupt the normal metabolic pathways of the fat cell; small drugs that kill cells (e.g., cancer drugs such as methotrexate, bromo-deoxyuridine, actinomycin D, nocodazole, brefeldin A, etc.); and peptides, small fragments of proteins that might prove to have functionality towards killing fat. Applications with DNA and RNA compounds can be preferred due to the recent completion of the human genome sequencing project, which holds the potential for researchers to identify the undoubtedly scores, if not hundreds, of molecules involved in fat regulation and metabolism. Other examples of the active fat-reducing substance are cytokine regulatory agents other than TNF-α, prolactin, beta-adrenergic stimulators and alpha-2 adrenergic inhibitors. These methods can be carried out analogously to those described for TNF-α. As can be seen, the ablation or elimination of the tissue can be effected by any of a number of mechanisms, including preventing its formation in the first place.

Additionally, the methods for delivery of TNF-α described herein can be readily modified for the treatment of other conditions characterized by excess tissue mass, particularly excess of tissue which is considered normally occurring (e.g., not cancerous tissue) but should be reduced or eliminated for health or cosmetic reasons. Examples of other tissues to which the inventive methods can be applied are pathologic hyperplasia, benign tumors, neointimal thickening of the vasculature, mole and hair removal. The method would be modified to use in place of the TNF-α an active substance effective for eliminating or preventing formation of cells of the unwanted tissue. An example of such other uses of the method relates to stem cells which have been under investigation for their ability to differentiate into mature tissue. A local, sustained release delivery according to this invention of factors that prevent the differentiation of stem cells could be used to prevent the restorative ability of the targeted tissue, resulting in reduction of that tissue.

Vehicles comprised of polymers, macromolecule-drug conjugates, hydrogels, etc., may be used as the sustained release carrier for delivering compounds that can target fat or other conditions where the end goal is to reduce tissue mass. Such materials and their preparation are known to those in the art. In the series of experiments described below, a polymer formulation consisting of poly(lactide-co-glycolide) (PLG) was used to deliver TNF-α. Other preferred polymers for the sustained release materials include but are not limited to poly(lactide)s, poly(glycolide)s, poly(lactic acid)s, poly(glycolic acid)s, polyanhydrides, polyorthoesters, polyetheresters, polycaprolactones, polyesteramides, polycarbonates, polycyanoacrylates, polyurethanes, polyacrylates, and blends or copolymers of the above. Among the preferred hydrogels for the sustained release materials are included optionally modified alginates, e.g., such as those disclosed in WO 98/12228, published Mar. 26, 1998. Among the preferred examples of macromolecule-drug conjugates are included macromolecules with contain polyethylene glycol groups for conjugation of the active substance. Preferably, the sustained release or controlled release materials are provided in the form of microparticles, such as microspheres, or as an injectable solution or gel. These preferably are relatively uniform in size and, for some embodiments, will can have a size of from 10 to 100 μm. Such materials can be provided and modified in known ways to control the rate of release in a manner which best facilitates the particular application of tissue reduction. For example, alginates and poly(lactide-co-glycolide)s can be provided as an injectable gel or processed into microspheres. For injectable solutions, a prepolymer solution can be injected which is then polymerized (e.g. by photopolymerization) or solidified (e.g., by using temperature sensitive gelling materials) in vivo. Preferably, the sustained release materials are selected to facilitate delivery of a substantially equal amount of the active substance per day, particularly over the course of from 3 days, more particularly at least 4 days, to over one year. Several rounds of injections can be made over time also to increase the effect.

The drugs which effect the tissue removal can be incorporated into the sustained release material by known methods. For example, by known double emulsion methods or, for hydrogels, by gelation crosslinking with cations. The amount of the drug incorporated into the sustained release material will be dependent on the nature of the sustained release material, the size of the sustained release material particles, the compatibility and releasability of the drug in such material, the amount of the drug desired to be delivered and the duration of the delivery, among others.

For methods of administering TNF-α in a PLG sustained release material to effect local fat tissue reduction, the particular parameters of administration would be designed to fit the particular circumstances. As non-limiting examples, it may be preferred that the PLG microspheres have a diameter of not larger than 100 □m, more preferably from 5 to 100 □m, and that TNF-α be incorporated in the PLG microspheres in an amount of 0.1 to 20% by weight. It may further be preferred that the formulation of drug and sustained release material provide effective release of the drug over a period of from 7 to 60 days.

Local administration of the sustained release formulations according to the invention is effected by injection directly into tissue to be eliminated or reduced, for example by subcutaneous or omental injection. In one embodiment, multiple such local injections of the sustained release formulations can be made at different points and/or depths of the tissue targeted for reduction. Preferably, such multiple injections are spaced such that a uniform reduction of the entire targeted tissue area is achieved.

In another embodiment, multiple active substances may be incorporated in a single controlled release material or in multiple controlled release materials. Such single controlled release material or multiple controlled release materials may be designed according to known methods (see, e.g., U.S. application Ser. No. 06/166,191; entitled, “Sustained drug delivery from structural matrices”, Shea, et al.) to release differing active substances at different times, e.g., sequentially, to facilitate removal of the unwanted tissue. An example of a useful application of sequential delivery is the delivery of anti-angiogenic compounds to destroy the blood supply, followed by the delivery of a molecule involved in inducing apoptosis (programmed cell death). Another example would be to destroy unwanted bone tissue by the administration of a drug that results in the demineralization of bone tissue followed by a molecule that kills the bone cells.

The methods for locally delivering substances (e.g., proteins, DNA, RNA, peptides, small molecule drugs) to remove tissue can also be used to remove such tissue in association with other types of tissue removal, e.g., surgical methods. Thus, the local delivery of the invention serves as an adjunct therapy to some other tissue removal method. For example, controlled release formulations of TNF-alpha can be locally administered to fat tissue to weaken the tissue and facilitate a following liposuction fat removal procedure. One example of the active substances which can be used to facilitate such weakening of the tissue for subsequent removal include molecules that inhibit extracellular matrix production. Examples thereof include: (1) collagenases and (2) inhibitors of collagen cross-linking, such as inhibitors of lysyl oxidase (a matrix crosslinking enzyme), e.g. beta-aminopropionitrile, or inhibitors of intermediate enzymes involved in the modifications of collagen prior to crosslinking. Further, other molecules may be delivered that chelate copper, a cofactor for lysyl oxidase's action. Another possibility is biological entities, such as bacteria (e.g., clostridium histolyticum from which some collagenases are derived) to weaken the tissue. Such treatment could be followed with delivery of another agent to kill the bacteria. Additionally, the patient's own cells could be used to weaken the tissue, for example T cells involved in inflammation to deliver the patient's own TNF-α or any of a number of peptides which elicit the inflammation response to recruit T cells to weaken the tissue.

The invention provides the first description of the use of sustained release polymers to engineer the local removal of smaller tissues from existing masses. The current state-of-the-art in tissue engineering centers on the creation of tissue utilizing the polymers described in this study. This novel approach, the use of these same polymer systems aimed at reducing tissue, represents an exciting advance in the application of these polymers.

The entire disclosure of all applications, patents and publications, cited above and below, and of U.S. Provisional Application No. 60/264,713, filed Jan. 30, 2001, are hereby incorporated by reference.

### BRIEF DESCRIPTION OF THE DRAWINGS

Various other features and attendant advantages of the present invention will be more fully appreciated as the same becomes better understood when considered in conjunction with the accompanying drawings, in which like reference characters designate the same or similar parts throughout the several views, and wherein:

FIG. 1 illustrates the sustained release profile of TNF-α from PLG microspheres in vitro.

FIG. 2 is a graph of cumulative TNF-α release for PLG microspheres in vitro over time

FIG. 3 is a graph of apoptic levels for PBS and blank microsphere controls and comparison of bolus injection of TNF-α protein and TNF-α released in a sustained manner.

FIG. 4 is a bar graph showing the continued fat ablation effect over time following TNF-α treatment according to the invention.

FIG. 5 is a bar graph showing the continued effects in blood levels of TNF-α, insulin and glucose over time following TNF-α treatment according to the invention.

### EXAMPLES

In the foregoing and in the following examples, all temperatures are set forth uncorrected in degrees Celsius; and, unless otherwise indicated, all parts and percentages are by weight.

### Example 1

PLG Microsphere Preparation

Microspheres were prepared by a double emulsion technique. A poly(lactide-co-glycolide) (PLG) (ratio of L:G was 75:25) was dissolved in ethyl acetate (first emulsion). TNF-α protein (10 μg) was dissolved in PBS (i.e., phosphate-buffered saline solution) (100 μl) and pipetted into 1 ml of the first emulsion, under the surface, and sonicated for 15 seconds to create the microspheres. The microsphere solution was mixed with an equal volume of 1% poly(vinyl alcohol)/7% ethyl acetate in deionized water, serving as the second emulsion, and vortexed vigorously for 15 seconds. The microsphere solution was placed in 0.3% poly-(vinyl alcohol)/7%ethyl acetate in deionized water (200 ml) and stirred vigorously until the organic component was completely evaporated. The microspheres were collected via filtration under vacuum through a 0.45 μm filter followed by a rinse with deionized water into a conical tube. The microspheres were frozen with liquid nitrogen and set to lyophilize until dry.

Release Kinetics from Microspheres

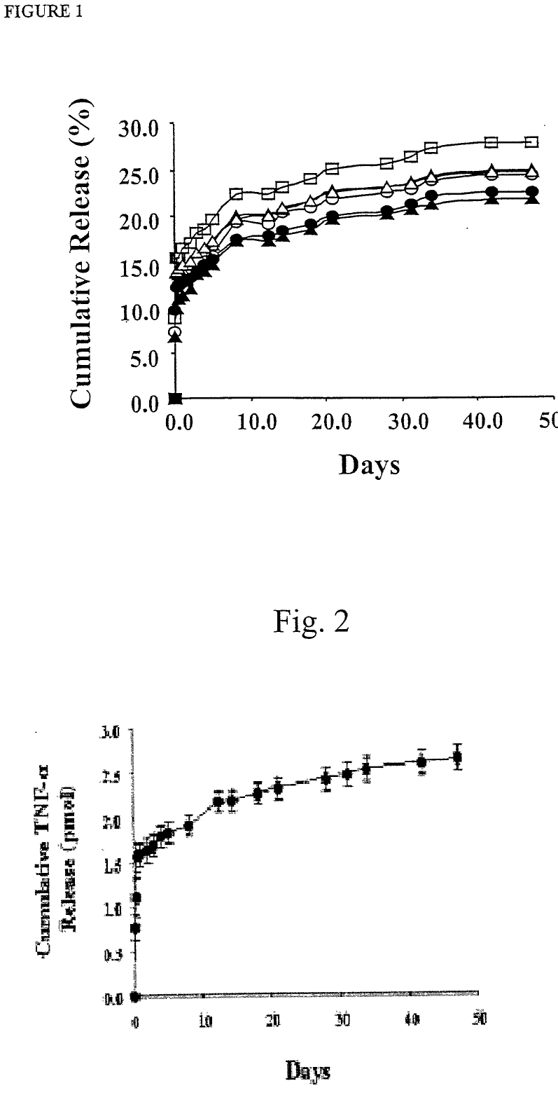

To demonstrate sustained release kinetics of encapsulated TNF-α, microspheres were prepared as described above with the exception that a small amount of radio-labeled factor (0.3 μCi/sample) was included as a tracer to quantify release. Microspheres (20 mg/sample) were re-suspended in 5 ml of PBS containing 0.1% Tween-20 at 25° C. The release of TNF-α was measured by the removal of the PBS, followed by counting in a gamma counter. Fresh PBS solution (with Tween) was added to the remaining microspheres. Counting was performed every day during the first week, every second day for the second week, etc., until 7 weeks of incubation, whereupon the remaining radioactivity in the microspheres was measured. The results of this test are shown in FIG. 1.

Diet Establishment

To provide functional adipose tissue, the following standard protocol was used. Sprague-Dawley rats (male, 8-10 weeks old) received either standard laboratory chow (Ralston-Purina, St. Louis, Mo.) containing 13% of calories from fat (4 rats) or a high-fat chow containing 35% lard (Bioserve, Frenchtown, N.J.) from which 55% of calories are derived from fat (4 rats). This protocol, using the Sprague-Dawley animal model, is widely used and has demonstrated a marked increase in weight. The animals were housed in a temperature-controlled facility, provided with food and water ad libitum, and maintained on a 12-hour light/dark cycle. Animals were weighed daily for 10 weeks and used for experiments.

In vivo Experiments

To demonstrate the ability of sustained, localized delivery of TNF-α, microspheres were injected into the rat epididymal fat pad and the tissue was monitored for mass changes. Microspheres were prepared as described above, containing TNF-α (1 or 25 μg), and resuspended in sterile PBS. Animals were briefly anaesthetized using Metophane. Microspheres were injected into one side of the epididymis, and blank microspheres containing no TNF-α were injected into the contralateral epididymis. To serve as a baseline measurement of epididymal fat pad mass, the fat pads from 2 animals were isolated and weighed. Animals were housed for 4 weeks post-injection, and the fat pads were removed from each animal and weighed.

Results

The data in FIG. 1 illustrate the sustained release profile of TNF-α from PLG microspheres in vitro. Following an initial burst period in the first few days, TNF-α exhibited essentially zero-order release kinetics. After about 7 weeks, about 25% of TNF-α was released. The data show release from 6 different samples of PLG (75:25).

The data in Table I illustrate the localized ablation of fat mass in vivo by the sustained delivery of TNF-α from the microspheres. Delivery of either 1 or 25 μg of TNF-α resulted in a drop of regional fat pad weight to about 85% that of the contralateral control tissue. To control for consistent fat pad isolation, normal diet rats were subjected to epididymal fat pad excision. The data demonstrate consistent fat pad isolation in which the relative standard deviation was only 10% (not shown). Interestingly, relatively small amounts of TNF-α (1 μg) were required to produce changes in the tissue. When a larger amount of TNF-α was injected, there was no significant change in the fat pad loss compared to the loss obtained with a smaller amount of TNF-α, indicating that the sustained release of a relatively small quantity of TNF-α can produce changes in fat pad mass. This also suggests that these is a saturation point in the process of fat ablation, indicating that the amount of the drug administered may not be as important as providing a sustained release of a smaller amount of drug over time.

TABLE I
Localized Reduction of Epididymal Fat Pad Mass by Sustained Delivery
of TNF-α
Effect of Sustained, Localized TNF-α Administration
(% of fat pad mass compared to control)

| | Fat Diet | Fat Diet | | | |
|---|---|---|---|---|---|
| | Rats given | Rats given | | | |
| | 25 μg | 1 μg | Fat Diet | Fat Diet | Fat Diet |
| | TNF-α | TNF-α | Rats given | Rats given | Rats given |
| | (4 week | (1 week | 1 μg TNF-α | 1 μg TNF-α | 1 μg TNF-α |
| | post- | post- | (4 week | (8 week | (12 week |
| | admin) | admin) | post-admin) | post-admin) | post-admin) |
| | 88.1 | 79.4 | 97.6 | 88.3 | 106.3 |
| | 81.2 | 76.2 | 78.6 | 101.7 | 87.6 |
| | 82.8 | 80.7 | 82.8 | 86.8 | 105.2 |
| | 85.7 | 77.5 | 90.5 | 84.6 | 72.7 |
| Aver- | 84.5 | 78.4 | 87.3 | 90.4 | 93.0 |
| age | | | | | |
| Std | 3.1 | 2.0 | 7.3 | 7.7 | 16.0 |
| Devi- | | | | | |
| ation | | | | | |

### Example 2

Tests were performed to verify the bioactivity of TNF-α in PLG microspheres following release by performing apoptosis staining of organ cultured adipose tissue. Further tests were performed on the ability of PLG-delivered TNF-α to decrease fat mass locally by injecting the microspheres into the epididymal fat pads of obese rats. Also, the functional localization of TNF-α was confirmed by performing blood analysis to measure systemic TNF-α, insulin and glucose levels.

Experimental Procedures

Materials

PLG [Resomer RG756 (75:25, i.v. 0.8 dl/g)] was purchased from Boehringer Ingleheim (Petersburg, Va.). Rat tumor necrosis factor-alpha was purchased from Intergen (Purchase, N.Y.), and ¹²⁵I-TNF-α was purchased from New England Nuclear (Boston, Mass.). Sprague-Dawley rats were purchased from Charles River Labs (Boston, Mass.). High fat diet rat chow, containing 55% of calories from fat, was purchased from BioServe (Frenchtown, N.J.). ELISA kits for rat TNF-α and rat insulin were purchased from R&D Systems (Minneapolis, Minn.) and CrystalChem (Chicago, Ill.), respectively. Glucose assay kits, Trinder-100, were purchased from Sigma (St. Louis, Mo.). The Bax detection kit used was the Catalyzed Signal Amplification purchased from DAKO (Carpinteria, Calif.), consisting of a primary rabbit polyclonal anti-human antibody, and secondary antibody biotinylated anti-rabbit IgG.

Microsphere Fabrication, Visualization and Analysis of Release Kinetics in vitro.

PLG microspheres were formed as previously described (26) with slight modification to incorporate TNF-α. Briefly, microspheres were fabricated using standard double emulsion processing containing 2 μg TNF-α per 50 mg polymer including (0.5 μCi) ¹²⁵I-TNF-α as a tracer in certain experiments. In vitro release kinetics from the polymer were performed (as described in Richardson, T. P. & Mooney, D. J. (2001) in Methods of Tissue Engineering, eds. Atala, A. & Lanza, R. (Academic Press, San Diego, Calif.), and Murphy, W. L., Peters, M. C., Kohn, D. H. & Mooney, D. J. (2000) Biomaterials 21, 2521-7) by quantifying the tracer released from the microspheres into PBS. Protein incorporation efficiency was determined to be 50-60%, resulting in 1 μg of TNF-α encapsulated per 50 mg microspheres. TNF-α microspheres were gold sputter-coated and visualized using a scanning electron microscope (Hitachi, S-3200N) at 4.5 keV.

Obese Rat Model

The treatment of experimental animals was in accordance with University of Michigan animal care guidelines, and we observed all NIH animal handling procedures. Sprague-Dawley rats (male, 8-10 weeks old) were maintained on high fat diets (55% of calories from fat) for three months to induce obesity. Microspheres containing TNF-α were injected into one epididymal fat pad of the obese rats (1 μg total of TNF-α, and blank microspheres containing no TNF-α served as an internal control for tissue mass loss. The animals were monitored for recovery for up to 12 weeks following microsphere injection, and weighed periodically. Epididymal fat tissue was excised and weighed at 0, 1, 4, 8, and 12 weeks (n=4 for each time point) following a single administration of TNF-α. Blood samples were taken from the orbital sinus at the noted time points, mixed with heparin, centrifuged and placed at −20° C. for later analysis.

Adipose Organ Culture

Epididymal fat tissue was excised and cut into pieces (1 cm³), and cultured. Briefly, tissue was placed in Dulbecco's Modified Eagle Medium containing 10% fetal bovine serum and penicillin/streptomycin, and incubated at 37° C. Samples (n=3) were injected with PBS, TNF-α protein (1 μg), blank microspheres, or microspheres containing TNF-α (total 1 μg TNF-α) and incubated at 37° C. After 3 or 7 days, the tissue was rinsed 1× with PBS, placed in 3.7% formaldehyde overnight and stored in 70% ethanol prior to sectioning for histology.

Histological Analysis

Tissue samples were bisected and subjected to butyl processing and paraffinization by standard procedures, at the histology core facility at the University of Michigan School of Dentistry. This process removes all lipids droplets from the tissue, enabling reproducible and rapid staining. Tissue sections were submitted to the Immunohistochemistry Core Facility at the University of Michigan, and stained with antibodies raised against Bax, a pro-apoptotic marker, to visualize the induction of apoptosis resulting from TNF-α treatment. Samples were counterstained with hematoxylin, imaged using a Nikon Eclipse E800, and Bax positive and negative cells were counted manually from at least 10 randomly chosen fields. Data were subjected to statistical analysis to determine the significance of the difference (P<0.05) among the measured population proportions of Bax positive cells, using a binomial equation.

Blood Analysis

Samples obtained from obese rats (n=4) undergoing TNF-α treatment were analyzed using ELISA kits for rat TNF-α and rat insulin levels in the serum, according to the manufacturers' instructions. Serum glucose levels were determined using glucose Trinder-100 kits according to manufacturer's instructions.

Results

TNF-α Delivery and Bioactivity

We fabricated microspheres from poly (lactide-co-glycolide) (PLG) for TNF-α delivery using a standard double emulsion processing technique previously demonstrated to allow localized delivery of proteins. This process allows for sustained protein delivery with rates controlled by polymer composition and molecular weight, enabling therapeutic versatility to optimize treatment and very low dosage requirements. PLG processed in this manner results in microspheres with diameters ranging between 5 and 60 μm, in which 80% measured 5-20 μm. We utilized PLG microspheres to encapsulate TNF-α, and these produced an in vitro TNF-α release rate of 1.6 pmol (81 ng) for the first day, followed by a sustained delivery rate of 0.023 pmol/day (1.2 ng) for the remainder of the study (FIG. 2). These systems can provide tailored delivery profiles (e.g., longer-term delivery, pulsatile delivery, extrinsically regulated), potentially enabling a wide utility for numerous applications if required.

We demonstrated the bioactivity of the released TNF-α by performing an apoptosis assay of adipocytes using an organ culture assay. After seven days incubation, control adipose in which phosphate-buffered saline (PBS) or microspheres (30mg) containing no TNF-α were injected revealed little baseline staining for Bax, a well-known proapoptotic protein. Positive controls with direct injection of TNF-α protein (equivalent mass to that encapsulated in PLG microspheres) into the tissue demonstrated high levels of staining. Tissues containing TNF-α releasing microspheres stained intensely for Bax, and the entire tissue lost integrity, for all samples treated with TNF-α from microspheres. TNF-α was further verified to be bioactive by performing apoptosis assays on cultured primary adipocytes and preadipocytes in vitro.

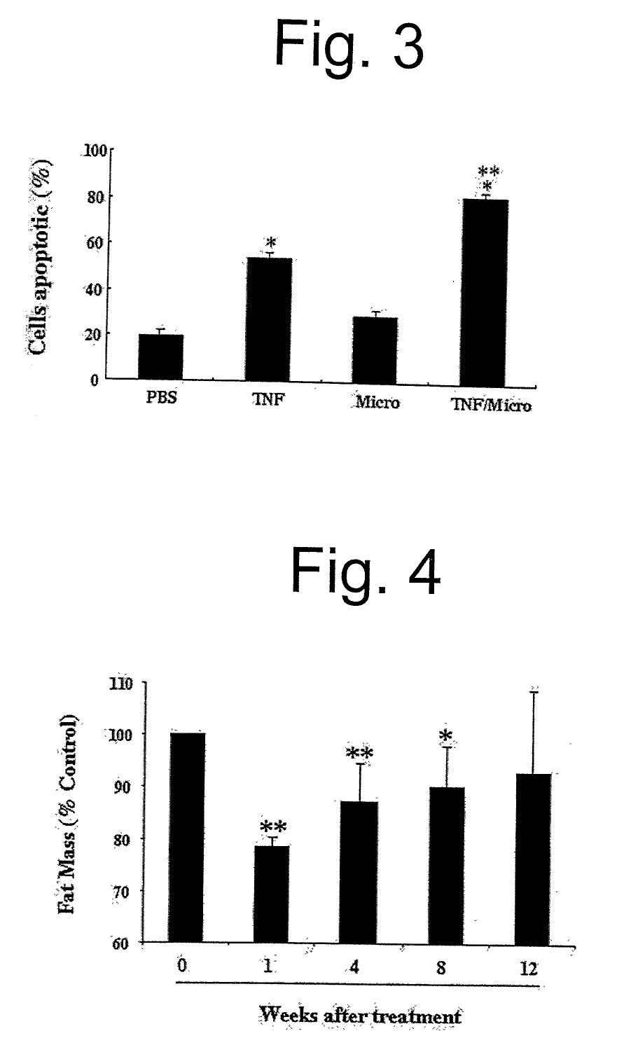

The apoptotic levels were quantified by manual analysis of the percentage of Bax positive cells in histological sections. Control conditions for both PBS and blank microspheres after seven days indicated baseline staining for Bax of 20% and 30%, respectively (FIG. 3), for the 1 cm³ explanted tissue. Bolus injection of TNF-α protein showed an induction of apoptotic cells (54%); TNF-α released in a sustained manner resulted in a much higher percentage, 82%, of the cells staining for apoptosis. After three days of incubation, the patterns were similar. These results indicate that TNF-α is active and maintains its normal physiologic role after encapsulation and release from PLG. Further, these data indicate that similar amounts of TNF-α can have markedly different effects on the apoptotic index of adipose tissue, based on the delivery method, and that sustained delivery is more effective than bolus administration.

Selective Adipose Ablation in vivo

To test the utility of the delivery system for selective localized tissue ablation, we utilized a standard rat model of obesity, the cafeteria-diet model in which 55% of calories derive from fat. We performed one injection of microspheres encapsulating TNF-α (1 μg) into the epididymal fat pad of obese Sprague-Dawley rats and blank microspheres containing no TNF-α into the contralateral fat pad to serve as a paired internal control. Animals were monitored for tip to 12 weeks. The weights of the excised fat pads showed a marked difference when treated with TNF-α. TNF-α treated fat pads, compared to the untreated controls, contained only 78% of the control adipose mass after one week (FIG. 4), a statistically significant difference (P<0.05). A significant mass difference between experimental and control conditions persisted throughout 8 weeks of the experiment (90%). After 12 weeks, though the animals were maintained on the high-fat diet, the mass of fat pads receiving drug-releasing microspheres remained at only 92.8% of the controls (P>0.10). These animals continued to gain weight and their phenotype was decidedly obese (weight in excess of 900 g; normal diet rats weighed 500-600 g), suggesting the local TNF-α delivery had no systemic effects. These results indicate that a single injection of 1 μg of TNF-α using sustained drug delivery can markedly alter adipose mass in specific depots even in the face of overall increases in obesity.

A striking observation in these studies was that delivery of TNF-α, both in vivo and in vitro, resulted in tissue with greatly decreased physical integrity (i.e., mechanically unstable during processing), indicative of large-scale cell death. After one week of treatment the tissue was dramatically weakened, consistent with marked TNF-α induced apoptosis. The in vitro adipose organ culture tissue was difficult to process for histological analysis, and similarly in vivo the epididymal fat pad lost much of its mechanical integrity.

Blood Analysis

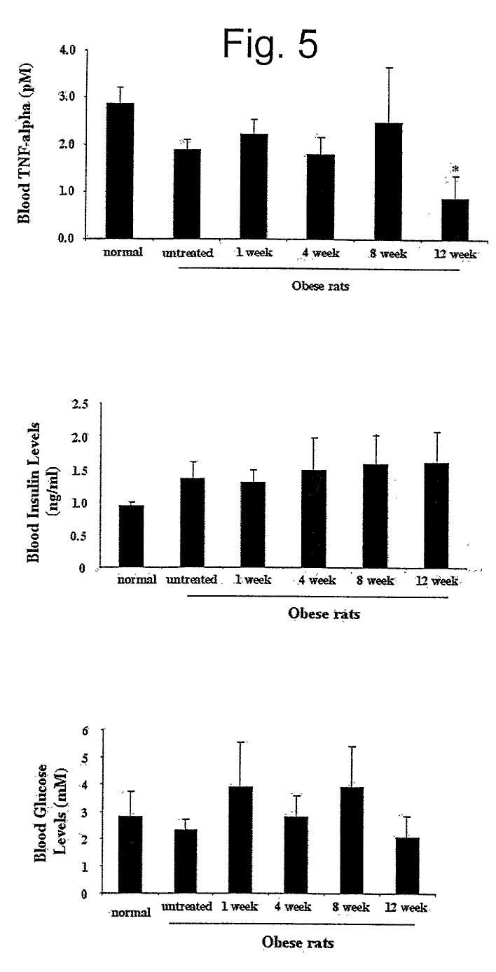

The negative effect of TNF-α on glucose metabolism has been very well documented and thus control over its localization and tissue availability is critical for optimal adipose ablation with minimal adverse metabolic effects. TNF-α localization in these studies was analyzed both directly and indirectly. First, enzyme-linked immunosorbent assays were used to measure blood serum levels of TNF-α. One week after injection with microspheres containing TNF-α, no significant differences in TNF-α levels were measured relative to normal diet, non-obese rats and high fat diet rats (P>0.05; FIG. 5A). TNF-α levels did not change until 12 weeks, when the animals were excessively obese, consistent with reports indicating a loss of endogenous TNF-α expression in morbidly obese subjects. These results indicate that low levels of protein delivered locally can have significant physiological effect without inducing increases in the systemic circulation.

We also analyzed systemic effects of TNF-α by measuring insulin levels in the serum following treatment with TNF-α encapsulated in microspheres. Systemic TNF-α elevates insulin levels due to the effect of TNF-α on glucose transport, resulting from the requirement of additional insulin to clear the glucose. After treatment for 1, 4, 8, or 12 weeks with microspheres containing TNF-α, the rats did not exhibit a statistically significant increase in insulin levels (P>0.05), relative to normal diet, non-obese rats and high fat diet rats. The basal insulin levels tended to rise with corresponding increases in obesity (FIG. 5B).

Lastly, we tested for potential systemic effects of locally administered TNF-α indirectly by measuring blood glucose levels. TNF-α, when present in the blood at sufficient concentrations, can induce adverse metabolic effects by rendering the host insensitive to insulin and thereby increasing blood glucose levels. We measured blood glucose in the animals, fed ad libitum, at each time point and found no significant difference at any time (FIG. 5C). These data indicate that the injection of TNF-α microspheres did not result in elevated glucose levels at these times (P>0.05), and that glucose levels remained in the normal physiologic range. Thus, locally delivered TNF-α had no effect on blood glucose or insulin.

Discussion of Examples

Demonstrated above is a novel injectable weight loss therapy that provides selective adipose tissue ablation. These drug delivery approaches demonstrate a selective loss of adipose tissue, for example, for Up to 12 weeks, using a well-known adipose tissue mass regulator. The concept that drug delivery approaches can be used for selective tissue ablation represents an attractive, minimally-invasive therapeutic modality having wide applicability for numerous indications where excess and unwanted tissue contribute to or cause disease and health risk.

Particularly, TNF-α, a well-known initiator of apoptosis in numerous cell types, is shown to be useful therapeutically for local fat ablation. While TNF-α was previously used to elicit weight loss (DeClerq, L., Genart, C., Boone, C. & Remacle, C. (1996) J Anim Sci 1996, 11), it was administered there systemically via a pump. Though effective for weight loss, systemic TNF-α induced a marked diabetic phenotype while present in the blood. Further, systemic TNF-α resulted in overall cachexia, and an anorexic phenotype in the animal models over time, demonstrating a lack of controlled and selective weight loss.

According to one embodiment of the current invention very low amounts of TNF-α (e.g., about 1 μg) can be locally delivered to effect adipose tissue ablation without inducing diabetic complications. The total amount of TNF-α delivered per day may be quite small and even if a small amount of the delivered TNF-α entered the systemic circulation its effect would likely be minimal. As expected, blood analysis revealed no significant increase in the amount of TNF-α present following microsphere delivery of TNF-α according to the invention, as determined by ELISA. Indirect evidence supporting this finding is that neither basal insulin nor glucose levels were elevated, a condition predicted to result from a diabetic phenotype caused by uncontrolled or systemic TNF-α delivery. The microspheres delivered 1.2 ng per day, indicating that polymeric protein delivery and protein-based therapy can provide significant physiological control with very low protein requirements. Taken together, these data indicate that localized, sustained delivery of TNF-α can elicit selective weight loss without significant systemic effects.

The in vitro experiments demonstrate that sustained delivery of TNF-α induced significantly higher levels of apoptosis, compared to bolus delivery of the same drug. Sustained TNF-α delivery was more effective at inducing apoptosis than bolus delivery, indicating that even lower doses might be effective if delivered in a sustained manner. Further, with low protein requirements, treatments involving several injections to various regions of excess adipose tissue are useful. This utility is augmented by the fact that protein delivery from PLG permits flexibility in dosing and time course for delivery, based on initial protein loading and the degradation and erosion profiles of the chosen polymer, which degrades over several months. This is an important factor for therapeutics based on proteins, which control complex biological processes that have distinct temporal and dose requirements for effective therapeutic outcome (i.e., tissue regeneration or reduction would be predicted to occur over weeks to months, while biochemical regulation, such as glucose clearance via insulin administration, would occur in hours).

Although not intending to be bound by this theory, it appears that the initial steps of adipose ablation are a weakening of the tissue following TNF-α administration. After TNF-α delivery, the tissue lost integrity, presumably due to TNF-α mediated apoptosis and lipolysis. The result is loss of cell number and a concurrent loss of lipid deposits in the resident adipocytes. Apparently, the associated lipolysis would result in lipids (triglycerides and free fatty acids) released into the blood, the extent of which would be related to a combination of the rates of adipose loss and of lipid clearance. The process of tissue resorption is presumably mediated in part by infiltration of the treated tissue by macrophages, cells containing high levels of TNF-α, which may actually aid in adipose tissue ablation. Significant inflammation, however, was not observed. Interestingly, while TNF-α has not been shown to initiate apoptosis through Bax directly, our data suggest that TNF-α may elicit an intracellular pathway utilizing Bax, an important member of the Bcl-2 family.

Adipose tissue ablation by locally delivered TNF-α is a powerful approach to ameliorate many of the health problems and risks associated with obesity. Administration of TNF-α may reduce adipose tissue to levels where the normal functioning of the tissue is restored (i.e., TNF-α expression may be regained) and the associated health risks are thereby minimized. Furthermore, as TNF-α induces apoptosis, it would be predicted that there would be a permanent loss of those adipocytes induced to undergo apoptosis, indicating that the potential to regain adipose mass is limited. In the above examples, the animals did not regain adipose mass to levels comparable to controls until 12 weeks after treatment although being maintained on a high fat diet, indicating that this effect is long term. Other drug candidates that could be similarly used for adipose tissue ablation include those that act directly on adipose tissue and the adipocytes to alter the differentiation status of the adipocytes (for example, altering PPAR-γ, C/EBPs, etc.; see, e.g., Brun et al., (1996) Curr. Opin. Cell Biol. 8, 826-832; Lofttis et al., (1997) Curr. Opin. Genet. Dev. 7, 603-608; Morrison et al., (1999) J. Cell Biochem. Suppl 32-33, 59-67; and Rangwala et al., (2000) Annu. Rev. Nutr. 20), those that interfere with the blood supply (anti-angiogenic therapy), or drugs that induce apoptosis and cell death (small drugs and other apoptotic inducers). Anti-obesity therapies would undoubtedly benefit from controlled delivery of drugs to specific depots associated with disease and disease-risk (e.g., visceral adipose depots).

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

We claim:

1. A method for eliminating or reducing normal but undesired tissue in a patient which comprises administering a controlled release formulation to the patient by injection at a local area containing the undesired tissue such that the undesired tissue in the local area is eliminated or reduced, said formulation comprising a substance which eliminates or prevents formation of the cells of the undesired tissue, said substance being provided in a controlled release carrier.

2. The method of claim 1 where the undesired tissue is fat tissue.

3. The method of claim 2 where the substance which eliminates or prevents formation of cells of the fat tissue is TNF-α.

4. The method of claim 2 where the substance which eliminates or prevents formation of cells of the fat tissue is a cytokine regulatory agent; a protein affecting fat metabolism; leptin; orexin; an antisense RNA molecule which knocks out the specific activity of a protein needed for fat cell maintenance; a DNA, either in the form of plasmid or virus, which induces the expression of apoptosis-inducing factors; a drug that kills fat cells; methotrexate; bromo-deoxyuridine; actinomycin D; nocodazole; brefeldin A; a peptide, having functionality which kills fat cells; prolactin; a beta-adrenergic stimulator; or, an alpha-2 adrenergic inhibitor.

5. The method of claim 1 where the controlled release carrier is comprised of a poly(lactide-co-glycolide) material.

6. The method of claim 2 where the controlled release carrier is comprised of a poly(lactide-co-glycolide) material.

7. The method of claim 1 where the controlled release formulation is injected multiple times distributed in the local area of the undesired tissue.

8. The method of claim 2 where the controlled release formulation is injected multiple times distributed in the local area of the undesired fat tissue.

9. The method of claim 1 where release of the substance which eliminates or prevents formation of cells of the undesired tissue is effected over at least 3 days by the controlled release carrier.

10. The method of claim 2 where release of the substance which eliminates or prevents formation of cells of the undesired fat tissue is effected over at least 3 days by the controlled release carrier.

11. The method of claim 9 where the substance which eliminates or prevents formation of cells of the undesired tissue is released in a substantially equal amount for each of the days of release.

12. The method of claim 10 where the substance which eliminates or prevents formation of cells of the undesired tissue is released in a substantially equal amount for each of the days of release.

13. The method of claim 1 where the undesired tissue is pathologic hyperplasia, benign tumor, neointimal thickened vasculature, mole or hair tissue.

14. The method of claim 3 where the controlled release carrier is comprised of a poly(lactide-co-glycolide) material.

15. The method of claim 14 where the TNF-α is provided in poly(lactide-co-glycolide) microspheres in an amount of from 0.1 to 20% by weight.

16. The method of claim 14 where the controlled release carrier provides in vivo release of the TNF-α for a period of 7 to 60 days.

17. The method of claim 1 where the controlled release carrier is comprised of a poly(lactide), poly(glycolide), poly(lactic acid), poly(glycolic acid), polyanhydride, polyorthoester, polyetherester, polycaprolactone, polyesteramide, polycarbonate, polycyanoacrylate, polyurethane, polyacrylate, blends or copolymer of the above polymers, a hydrogel, an alginate or modified alginate, or a polyethylene glycol group-containing macromolecule for conjugation of the active substance.

18. A method for eliminating or reducing normal but undesired tissue in a patient which comprises administering a controlled release formulation, said formulation comprising a substance which weakens or hinders formation of the undesired tissue provided in a controlled release carrier, to the patient by injection at a local area containing the undesired tissue such that removal of the undesired tissue by other means is enhanced and conducting the other means of removal.

19. The method of claim 18 where the tissue is fat tissue and the other means of removal of the tissue is liposuction.

20. The method of claim 18 wherein the substance which weakens or hinders formation of the undesired tissue is a substance which inhibits extracellular matrix production.

21. The method of claim 20 wherein the substance is a collagenase or an inhibitor of a collagen cross-linking enzyme.

22. The method of claim 20 where the substance is an inhibitor of lysyl oxidase.

23. The method of claim 20 wherein the substance is beta-aminopropionitrile.

24. The method claim 1 wherein the formulation comprises two or more substances in the controlled release carrier having a combined action of eliminating or preventing formation of the cells of the undesired tissue.

25. The method of claim 24 wherein at least one of the substances is released from the controlled release carrier later in time than another of the substances.

26. The method of claim 25 wherein a first substance released is an anti-angiogenic compound which hinders the blood supply to the unwanted tissue and a second substance is released later in time which induces apoptosis in the unwanted tissue.

27. The method of claim 25 for removing unwanted bone tissue wherein a first substance is released which demineralizes the bone tissue and a second substance is released later in time which kills cells of the bone tissue.

28. The method of claim 18 wherein the formulation comprises two or more substances in the controlled release carrier having a combined action of weakening or hindering formation of the undesired tissue.

29. The method of claim 28 wherein at least one of the substances is released from the controlled release carrier later in time than another of the substances.

30. The method of claim 29 wherein a first substance released is an anti-angiogenic compound which hinders the blood supply to the unwanted tissue and a second substance is released later in time which induces apoptosis in the unwanted tissue.

31. The method of claim 29 for removing unwanted bone tissue wherein a first substance is released which demineralizes the bone tissue and a second substance is released later in time which kills cells of the bone tissue.
